(19) United States
(12) Patent Application Publication
(10) Pub. No.: US 2024/0299205 A1
(43) Pub. Date: Sep. 12, 2024

(54) External Catheter with Finger Pocket for Self-Placement

(71) Applicant: C. R. Bard, Inc., Franklin Lakes, NJ (US)

(72) Inventor: James David Hughett, SR., Monroe, GA (US)

(21) Appl. No.: 18/664,024

(22) Filed: May 14, 2024

Related U.S. Application Data
(63) Continuation of application No. 17/528,037, filed on Nov. 16, 2021.
(60) Provisional application No. 63/115,564, filed on Nov. 18, 2020.

(51) Int. Cl. A61F 5/455; A61M 1/00; A61M 25/01

(52) U.S. Cl. CPC A61F 5/455; A61M 1/60; A61M 1/64; A61M 25/0111; A61M 2210/1092

(57) ABSTRACT

A female external catheter includes a hollow body portion including a urethral inlet in fluid communication with an outlet. A wicking material covers the urethral inlet, and a catheter tube is coupled to the outlet, the catheter tube configured to transport urine away from the hollow body portion. The hollow body portion includes a finger portion with an opening configured to receive only a single finger to enable manipulation of the female external catheter in order to position and hold the wicking material against a urethral outlet of a female user.

### PRIORITY

This application is a continuation of U.S. patent application Ser. No. 17/528,037, filed Nov. 16, 2021, which claims the benefit of priority to U.S. Provisional Application No. 63/115,564, filed Nov. 18, 2020, each of which is incorporated by reference in its entirety into this application.

### BACKGROUND

For reasons of timeliness, convenience, or modesty, patients often prefer self-placement of external catheters over clinician-placement thereof as needed to void their bladders. In view of the foregoing, external catheters for self-placement are needed.

Disclosed herein are external catheters for self-placement in view of at least the foregoing need.

### SUMMARY

Briefly summarized, embodiments disclosed herein are directed to systems, methods and apparatuses for self-placing an external catheter by a patient. One problem that often arises with external catheters is the need for a caregiver to place the catheter on the patient. In particular, concerns may arise regarding patients' bodily privacy. For example, some patients feel ashamed or embarrassed by requiring aid from a caregiver in order to place a catheter of the current technology, such as urinary catheter 110 of FIG. 1. Such patients need a method to place and remove the catheter for themselves. A second problem is that patients who are able to control the timing of when they urinate but still need assistance urinating, may wish to remove and dispose of their urinary catheter when it is not needed. Similarly, some patients are able to control the timing of when they urinate, but have limited mobility and have difficulty walking to a restroom. Accordingly, some patients may only need to use the catheter actively for circumscribed periods, such as a few hours per day or less. Moreover, after use, a urinary catheter can become soiled, so it can be important to remove the catheter for reasons of cleanliness, sanitation, and comfort.

Disclosed herein is a self-placeable external catheter. In some embodiments, the external catheter comprises a catheter body having at least a top side and a bottom, a wicking area disposed on the top side, a finger covering extending from the bottom side of the catheter body creating a cavity configured to receive a user finger and an aperture formed in the bottom side of the external catheter, wherein the wicking area receives urine such that the urine passes through the wicking area, through the catheter body and through the aperture.

Catheter tubing may be inserted into the aperture such that the urine passes through the wicking area, through the catheter body and through the aperture into the catheter tubing. In some embodiments, a raised ridge surrounds the aperture. In other embodiments, an opening of the cavity faces a proximal side of the catheter body.

In some embodiments, the aperture is located at a distal side of the catheter body. In some embodiments, the external catheter further comprises a raised, curved surface located at a proximal end of the top side of the catheter body, wherein the raised, curved surface is adjacent to the wicking area. The finger covering may fully enclose a distal side of the cavity. The external catheter may further comprise catheter tubing, wherein a proximal end of the catheter tubing extends from the aperture and a collection container, wherein the urine is collected in the collection container. Additionally, the external catheter may further comprise a suction pump coupled to a distal end of the catheter tubing, wherein the suction pump provides suction that draws the urine toward the collection container. In some embodiments, the wicking area is comprised of gauze.

Also disclosed is a method of placing a self-placeable external catheter comprising at least a first step of inserting, by a user, a finger into a cavity of the external catheter, wherein the external catheter includes a catheter body having at least a top side and a bottom side, a wicking area disposed on the top side, a finger covering extending from the bottom side of the catheter body creating the cavity configured to receive the finger, an aperture formed in the bottom side of the external catheter, wherein the wicking area receives urine such that the urine passes through the wicking area, through the catheter body and through the aperture, and a second step of placing the external catheter such that the wicking area of the external catheter is in contact with a urethra of a patient. In some embodiments, the user and patient is female. Additionally, the method may comprise an additional step of initiating operation of a suction pump which creates suction to draw urine from the patient toward a collection container.

These and other features of the concepts provided herein will become more apparent to those of skill in the art in view of the accompanying drawings and following description, which disclose particular embodiments of such concepts in greater detail.

### BRIEF DESCRIPTION OF DRAWINGS

Embodiments of the disclosure are illustrated by way of example and not by way of limitation in the figures of the accompanying drawings, in which like references indicate similar elements and in which:

FIG. 1 illustrates an external urinary catheter placed in a patient, according to some embodiments;

FIG. 2 illustrates usage of a self-placeable external catheter with a finger pocket, according to some embodiments;

FIG. 3A shows a perspective view of a self-placeable external catheter with a finger pocket, according to some embodiments;

FIG. 3B shows a second perspective view of a self-placeable external catheter with a finger pocket, according to some embodiments;

FIG. 3C shows a second embodiment to of the self-placeable external catheter with a finger pocket of FIGS. 3A-3B, according to some embodiments; and

FIG. 4 displays a flowchart of an example method for using a self-placeable external catheter with a finger pocket, according to some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal end portion” of, for example, a probe disclosed herein includes a portion of the probe intended to be near a clinician when the probe is used on a patient. Likewise, a “proximal length” of, for example, the probe includes a length of the probe intended to be near the clinician when the probe is used on the patient. A “proximal end” of, for example, the probe includes an end of the probe intended to be near the clinician when the probe is used on the patient. The proximal portion, the proximal end portion, or the proximal length of the probe can include the proximal end of the probe; however, the proximal portion, the proximal end portion, or the proximal length of the probe need not include the proximal end of the probe. That is, unless context suggests otherwise, the proximal portion, the proximal end portion, or the proximal length of the probe is not a terminal portion or terminal length of the probe.

With respect to “distal,” a “distal portion” or a “distal end portion” of, for example, a probe disclosed herein includes a portion of the probe intended to be near or in a patient when the probe is used on the patient. Likewise, a “distal length” of, for example, the probe includes a length of the probe intended to be near or in the patient when the probe is used on the patient. A “distal end” of, for example, the probe includes an end of the probe intended to be near or in the patient when the probe is used on the patient. The distal portion, the distal end portion, or the distal length of the probe can include the distal end of the probe; however, the distal portion, the distal end portion, or the distal length of the probe need not include the distal end of the probe. That is, unless context suggests otherwise, the distal portion, the distal end portion, or the distal length of the probe is not a terminal portion or terminal length of the probe.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Additionally, or in the alternative, the term logic may refer to or include software such as one or more processes, one or more instances, Application Programming Interface(s) (API), subroutine(s), function(s), applet(s), servlet(s), routine(s), source code, object code, shared library/dynamic link library (dll), or even one or more instructions. This software may be stored in any type of a suitable non-transitory storage medium, or transitory storage medium (e.g., electrical, optical, acoustical or other form of propagated signals such as carrier waves, infrared signals, or digital signals). Examples of a non-transitory storage medium may include, but are not limited or restricted to a programmable circuit; non-persistent storage such as volatile memory (e.g., any type of random-access memory “RAM”); or persistent storage such as non-volatile memory (e.g., read-only memory “ROM”, power-backed RAM, flash memory, phase-change memory, etc.), a solid-state drive, hard disk drive, an optical disc drive, or a portable memory device. As firmware, the logic may be stored in persistent storage.

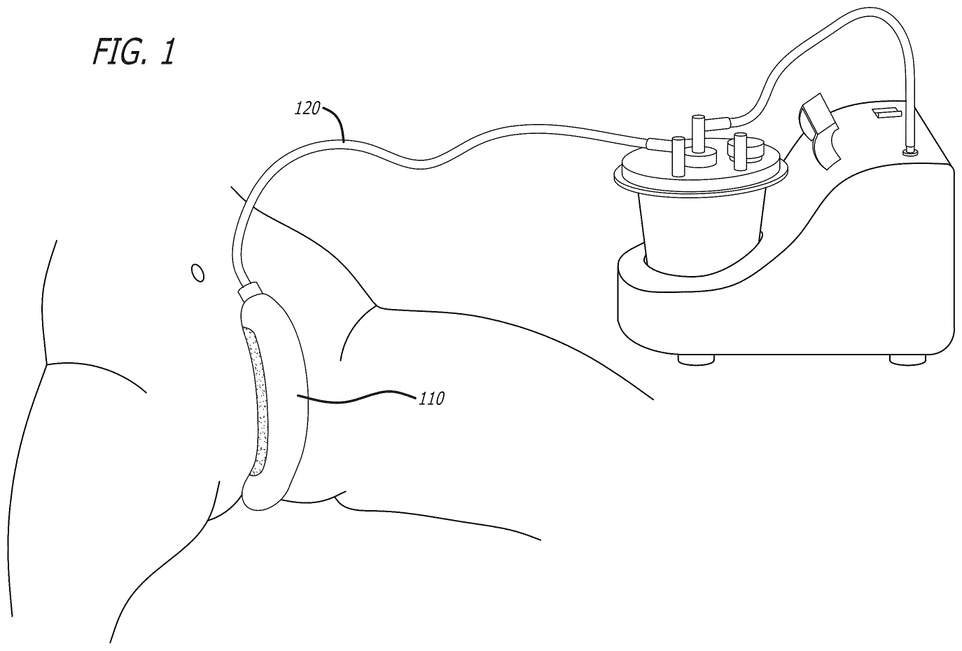

FIG. 1 illustrates an external urinary catheter of current technology placed in, on or adjacent to a patient, according to some embodiments. Urinary catheter 110 can be part of a system for patients with bladder incontinence, which can also include catheter tubing 120, an electric pump, and a canister assembly to collect the patient's urine. In this example, urinary catheter 110 is inserted over the urethra of a patient with bladder incontinence. Urinary catheter 110 is connected to catheter tubing 240, which leads to a canister assembly (not shown). Urinary catheter 110 can gently “wick” urine away from the patient, i.e., remove the urine by capillary action. An electric pump (not shown) can pump the patient's urine through catheter tubing 240 and to the canister. The canister can be removed and cleaned while not in use. For patients with bladder incontinence, the system of urinary catheter 110, the pump, and the canister can provide a safe, convenient, and effective way to catheterize.

Some patients cannot predict when they will urinate, and may actively use urinary catheter 110 for extended periods throughout the day. However, many patients are able to control the timing of when they need to urinate. Such patients may still need assistance urinating, and therefore still use urinary catheter 110 when they urinate. Similarly, some patients may be able to control the timing of when they urinate, but have limited mobility and have difficulty walking to a restroom, and therefore still use urinary catheter 110.

Such patients may wish to remove and dispose of urinary catheter 110 when it is not needed. In particular, such patients may only need to use urinary catheter 110 actively for relatively brief periods of time, such as a few hours per day or less. Moreover, urinary catheter 110 can become soiled after use, and it can be important to remove urinary catheter 110 for reasons of cleanliness, sanitation, and comfort. Even if the patient uses urinary catheter 110 continuously, it must be replaced periodically with a clean catheter. The disclosed system and methods can address these concerns.

Typically, urinary catheter 110 must be placed by a caregiver in the patient. When placed properly, urinary catheter 110 can adhere to the patient's labia, while a wicking area can make close contact with the patient's urethra, thereby allowing the urinary catheter 110 to wick urine efficiently from the patient. Placing urinary catheter 110 properly requires some technique, and moreover a patient cannot reach in order to place urinary catheter 110 herself. Accordingly, urinary catheter 110 may be placed by a caregiver, such as a home health aide, a nurse, a relative, or a spouse.

However, many patients are concerned about bodily privacy. These patients may feel ashamed or embarrassed to require aid from a caregiver in order to place urinary catheter 110. Such patients need a method to place and remove urinary catheter 110 for themselves. For such patients, the disclosed system and methods can address their desire for privacy.

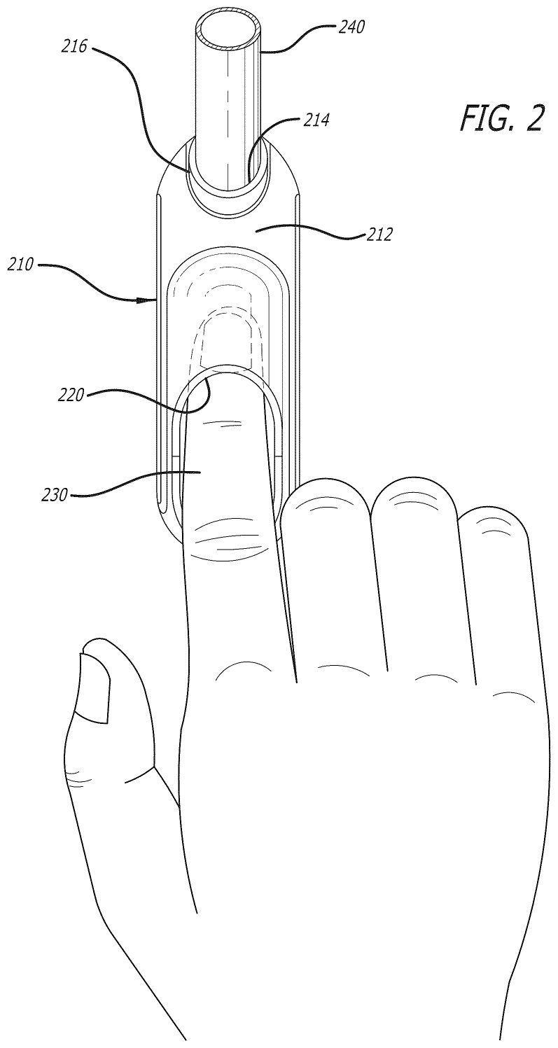

Referring to FIG. 2, usage of a self-placeable external catheter 210 with a finger pocket 220 is illustrated, according to some embodiments. In this example, a user mounts external catheter 210 on her finger 230. In a typical case, the user may be the patient, who wishes to self-catheterize. Alternatively, the user may be a caregiver, such as a home health aide, a nurse, a relative, or a spouse.

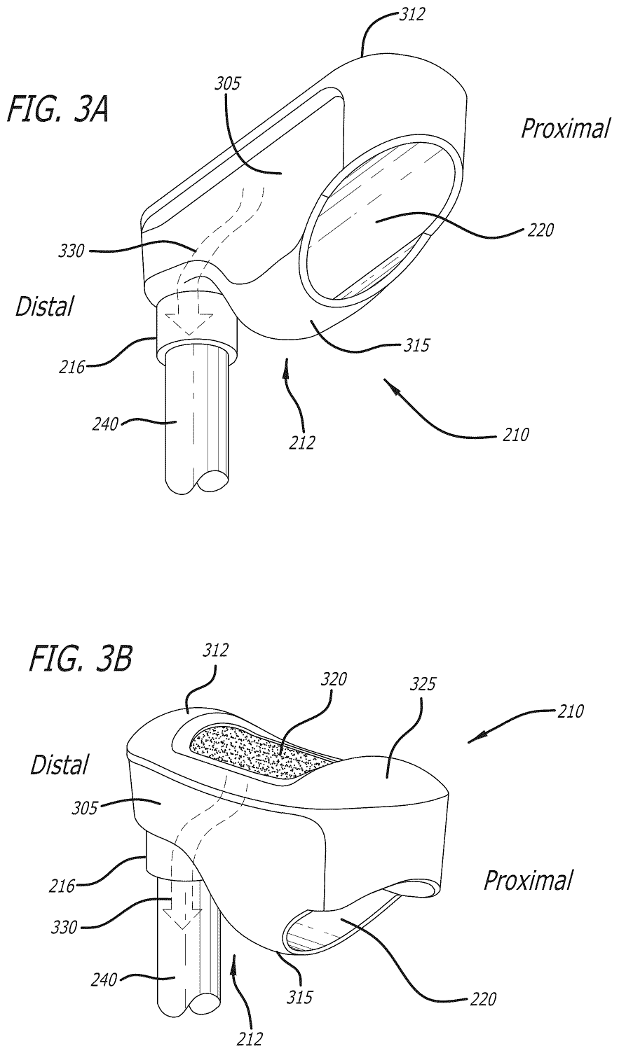

In this example, the user inserts her finger 230 in the finger pocket 220 with the palmar side of the finger 230 facing away from a bottom side 212 of the external catheter 210. The finger pocket 220 is located on the bottom side 212 of the external catheter 210. The finger pocket 220 may be a cavity that is configured to receive the user's finger 230. Specifically, once the user has inserted her finger 230 into the finger pocket 220, she may manipulate the placement and orientation of the external catheter 210 such that a wicking area (e.g., wicking area 320 of FIG. 3B) is placed over the patient's urethra in order to collect or “wick” urine, which is passed to the catheter tubing 240. In some embodiments, the wicking area is comprised of gauze.

As best seen in FIGS. 3A-3B, the catheter tubing 240 connects to the external catheter 210 via an aperture 214 also located on the bottom side 212 of the external catheter 210. Specifically, the aperture 214 may be located at a distal end portion, which, as shown, is located opposite the opening of the finger pocket 220. In some embodiments, the aperture 214 may be surrounded by a raised ridge 216. The external catheter 210 is configured to receive the catheter tubing 240, e.g., the catheter tubing 240 is inserted into the aperture 214. Due to the sizing of the aperture 214, insertion of the catheter tubing 240 requires particular force. The friction created between the catheter tubing 240 and the external catheter 210, specifically, the ridge 216, maintains the coupling of the two components.

Because finger pocket 220 can fasten to the user's finger 230, it can enable a patient to reach more efficiently and effectively, thereby placing external catheter 210 for herself. In this example, the external catheter 210 with finger pocket 220 may extend beyond the user's fingertip, thereby extending the user's reach. Moreover, by clasping the user's finger 230, finger pocket 220 can free most of the user's fingers to position external catheter 210 and to control its placement, rather than simply holding external catheter 210. Accordingly, finger pocket 220 makes it possible for a patient to place external catheter 210 in her own body in private, without assistance from a caretaker. By enabling the patient to self-catheterize, the disclosed catheter, system, and methods can greatly increase the convenience of using external catheter 210, as well as expand its utility to a significantly broader class of patients.

Referring now to FIGS. 3A-3B, two perspective views of a self-placeable external catheter 210 with a finger pocket 220 are shown according to some embodiments. The external catheter 210 is shown to comprise a hollow body portion 305 where the finger pocket 220, e.g., a cavity, is created on the bottom side 212 of the body portion 305 as a finger covering 315 extends away from the body portion 305. In this view, the catheter tubing 240 is located on the distal end portion on the bottom side 212 of the external catheter 210.

In various embodiments, finger pocket 220 can be any opening or groove that provides space for a user's finger to be inserted into external catheter 210, or provides a convenient or efficient method of gripping or handling external catheter 210. In particular, finger pocket 220 enables the user to manipulate, position, and hold external catheter 210 stably without needing to engage her entire hand. For example, the user can manipulate and control external catheter 210 using just one finger, such as by coupling external catheter 210 to the user's index finger with finger pocket 220, as in the example of FIG. 2 above.

The wicking area 320, which is to be placed over the patient's urethra, is located on the top surface 312 of the external catheter 210. In some embodiments, the wicking area 320 may be centrally located between the distal and proximal end portions. During usage, i.e., when the wicking area 320 is placed over the urethra, the wicking area 320 receives urine 330 of the patient. The received urine 330 flows through the wicking area 320, passes through the hollow body portion 305 and is directed to the catheter tubing 240.

The external catheter 210 may also include a raised, curved surface 325 at a proximal end, where the raised, curved surface 325 may serve as an indicator of the placement of the wicking area 320 to the user as the user is attempting to place the wicking area 320 on her urethra. The placement of the wicking area 320 may be understood based on the user's knowledge of the location of the wicking area 320 in relation to the location of the raised, curved surface 325. In one example, as the external catheter 210 may be used discreetly, the user may not be able to obtain a visual of the placement of the wicking area 320. Therefore, in order to ensure proper placement, the raised, curved surface 325 may provide an indication as to placement when the external catheter 210 is placed against the user's body. In some embodiments, although not shown, the raised, curved surface 325 may include ridges, raised dots or other markings that may provide additional notice to the user as to the location of the wicking area 320. Referring to FIG. 3B, the wicking area 320 is shown to have a curved, convex outer surface. However, the wicking area 320 may have a flat, or substantially flat outer surface or have a curved, concave outer surface.

Referring now to FIG. 3C, in an alternative embodiment to FIGS. 3A-3B, the finger covering may not form a fully enclosed cavity (e.g., finger pocket 220) but, instead, the finger covering 340 be formed as a strip or strap leaving an opening on a distal side.

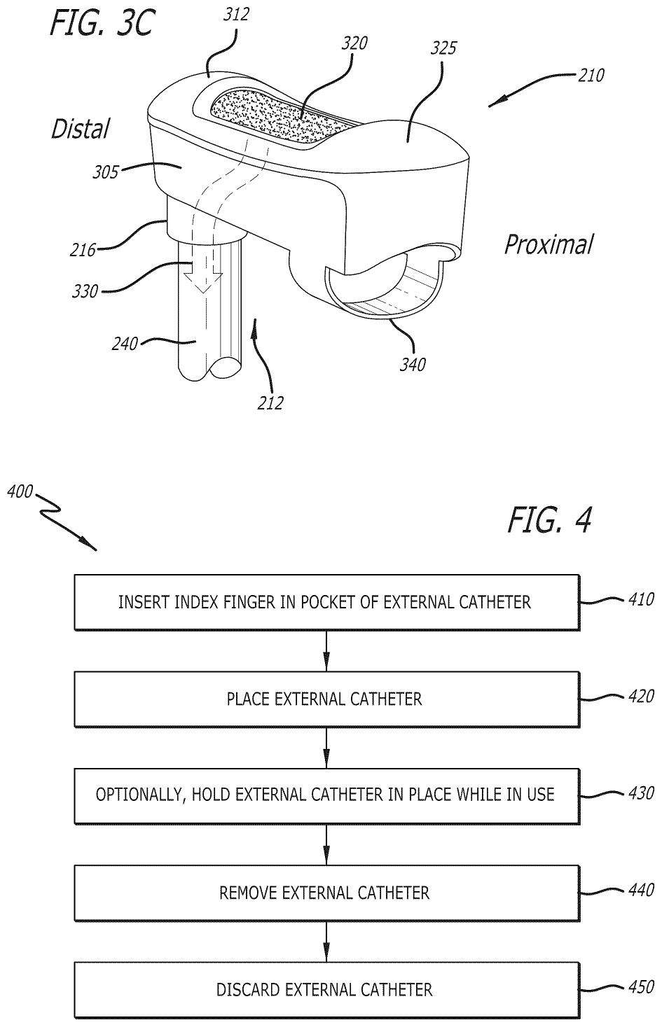

FIG. 4 displays a flowchart of an example method 400 for placing and using a self-placeable external catheter with a finger pocket, according to some embodiments. Each block illustrated in FIG. 4 represents an operation performed in the method 400 of placing and using the external catheter with a finger pocket of FIGS. 3A-3B. In an embodiment, method 400 is performed by the patient, who places the catheter on herself. The method 400 can also be performed by a caregiver or another person, and is not limited by the present disclosure. Accordingly, for generality, method 400 will be referred to herein as performed by a user, who may be the patient or another person.

As an initial step in the method 400, the user's finger is inserted into the pocket of the external catheter (block 410). In a typical example, the finger may be the user's index finger with the palmer side of the finger facing away from the bottom side of the catheter. In various embodiments, the finger pocket can include a cavity or other groove into which the user's finger is inserted.

Next, the external catheter can be placed on the patient (block 420). The catheter with finger pocket may extend the user's reach and/or free most of the user's fingers to control the catheter's placement. Thus, using the disclosed finger pocket, a patient can place the catheter on her own body, without needing assistance from a caregiver. Specifically, the user places a wicking area of the external catheter over the patient's urethra, so that, as urine is excreted, the urine is wicked through the wicking area, flows through the catheter body and is directed into the catheter tubing, which terminates at a urine collection container to collect the urine. In some embodiments, the urine collection container may be a pump container (which may be coupled with a pump) or may be a urine collection bag, i.e., strapped to the patient's leg.

Placing the catheter correctly requires close contact between the wicking area and the patient's urethra. In an embodiment, a raised, curved surface of the catheter, which may be located adjacent to the wicking area, may give the user an indication that the catheter is in proper positions. Next, the external catheter is held in place while in use, e.g., the patient is urinating (block 430). In particular, if the patient can control the timing of when she urinates, the patient will likely carry out the step of block 430, i.e., hold the catheter in place while it is in use. In this case, the patient can start and stop self-catheterizing whenever convenient. Moreover, by facilitating placement of the catheter, the disclosed finger pocket allows the patient to self-catheterize in complete privacy.

Next, the external catheter can be removed from the patient's urethra after usage, e.g., the patient has finished urinating (block 440). In an embodiment, the patient can remove the external catheter with one hand. For example, as the user has held the external catheter in place during usage, the user can remove the catheter shortly after urinating by simply moving her finger while the catheter remains fastened to it. Finally, the external catheter can be discarded (block 450). Because the catheter can become soiled, for reasons of cleanliness, sanitation, and comfort may important to remove and discard the catheter after use.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A female external catheter, comprising:
a hollow body portion including a urethral inlet in fluid communication with an outlet;
a wicking material covering the urethral inlet; and
a catheter tube coupled to the outlet, the catheter tube configured to transport urine away from the hollow body portion,
wherein the hollow body portion includes a finger portion with an opening configured to receive only a single finger to enable manipulation of the female external catheter in order to position and hold the wicking material against a urethral outlet of a female user.

2. The female external catheter according to claim 1, wherein the hollow body portion includes a raised ridge surrounding the outlet.

3. The female external catheter according to claim 1, wherein a surface surrounding the urethral inlet includes a raised and curved contour to enable proper positioning of the female external catheter.

4. The female external catheter according to claim 3, wherein the surface surrounding the urethral inlet further comprises additional features selected from the group consisting of a plurality of ridges, a plurality of raised dots, and combinations thereof.

5. The female external catheter according to claim 3, wherein the raised and curved contour is convexly shaped.

6. The female external catheter according to claim 1, wherein a surface surrounding the urethral inlet is substantially flat.

7. The female external catheter according to claim 1, wherein the finger portion is covered along a length of the hollow body portion.

8. The female external catheter according to claim 1, wherein the finger portion is ring-shaped.

9. The female external catheter according to claim 1, further comprising a collection container coupled to the catheter tube.

10. The female external catheter according to claim 9, further comprising a suction pump coupled to the collection container.

11. The female external catheter according to claim 1, wherein the wicking material is comprised of gauze.
